(19) United States
(12) Patent Application Publication
(10) Pub. No.: US 2023/0001217 A1
(43) Pub. Date: Jan. 5, 2023

(54) INDUCTIVE TRANSCUTANEOUS POWER DEVICE WITH OPEN-LOOP TEMPERATURE CONTROL

(71) Applicant: INCUBE LABS, LLC, San Jose, CA (US)

(72) Inventor: Mir A. IMRAN, Los Altos Hills, CA (US)

(73) Assignee: INCUBE LABS, LLC, San Jose, CA (US)

(21) Appl. No.: 17/781,589

(22) PCT Filed: Dec. 2, 2020
(86) PCT No.: PCT/US2020/062789

§ 371 Date: Jun. 1, 2022

Related U.S. Application Data
(60) Provisional application No. 62/943,706, filed on Dec. 4, 2019.

(51) Int. Cl. A61N 1/372; H01M 10/658; C01B 33/159

(52) U.S. Cl. CPC A61N 1/37229; H01M 10/658; C01B 33/159

(57) ABSTRACT

An apparatus is provided for remotely powering an implantable medical device (IMD) positioned at a target treatment location within a patient. The apparatus is configured to be positioned at or near an external skin surface of the patient in proximity to the target treatment location. The apparatus includes an induction coil which, when placed in proximity to the IMD, forms an inductive transcutaneous power link with the IMD such that when the induction coil is supplied with a current, the induction coil inductively and transcutaneously delivers power to the IMD. An aerogel layer is disposed between the induction coil and the patient's skin surface. The aerogel layer is configured to receive heat generated from the induction coil and regulate heat dissipation from the aerogel layer to minimize heat transfer to the patient.

### CROSS-REFERENCES TO RELATED APPLICATIONS

This application claims priority to, and the benefit of, U.S. Provisional Patent Application No. 62/943,706 filed on Dec. 4, 2019 and titled INDUCTIVE TRANSCUTANEOUS POWER DEVICE WITH OPEN-LOOP TEMPERATURE CONTROL, which is incorporated herein by reference in its entirety for all purposes.

### BACKGROUND

### Technical Field

Embodiments of the present description relate to implantable medical devices. More specifically, embodiments of the present description relate to an external charging device for powering an implantable medical device.

### Background Discussion

Medical devices having one or more implantable components, generally referred to as implantable medical devices (IMDs), may be used to monitor a patient's condition and/or to deliver therapy to the patient. In particular, devices such as electrical stimulation or treatment devices, implantable pacemakers, defibrillators, hearing aids, organ assistant or replacement devices, and other partially or completely implanted medical devices have been successful in performing lifesaving and/or lifestyle enhancing functions for a number of years.

Often, an external component or device is employed to provide power to, and/or to communicate with, the IMD. In long term or chronic uses, IMDs may include a rechargeable power source (e.g., including one or more capacitors or batteries) that extends the operational life of the medical device to weeks, months, or even years longer than a non-rechargeable device. When the energy stored in the IMD rechargeable power source has been depleted, the patient may recharge the power source via the external component, which is typically placed adjacent to or near the patient's skin. Since the rechargeable power source is implanted in the patient and the charging device is external to the patient, this charging process may be referred to as transcutaneous (through the skin) charging. In some examples, transcutaneous charging may be performed via inductive coupling between a primary coil in the external component and a secondary coil in the IMD, the external component and the IMD thus acting in concert to form an inductive transcutaneous power link.

One drawback to such closed-loop inductive coupling is that the external component often generates heat, which can cause discomfort or otherwise harm a patient or damage a patient's apparel. Improvements have been achieved by use of closed-loop sensing systems that monitor the temperature of the external component and modulate or stop operation of the inductive coupling based on feedback from a sensor. However, this results in additional potential failure modes and overly complicates and adds cost to the system. Also, efficiency may be reduced as a result of modulation of the inductive coupling resulting from the closed-loop control.

Thus, there is a need for providing a mechanism to improve temperature control in these types of biomedical devices.

### SUMMARY

The technology of the present disclosure is directed to systems and methods for providing an external inductive transcutaneous power link with open-loop temperature control for powering/charging an IMD or providing for communication with an IMD via inductive coupling with the IMD.

In one aspect of the technology of the present disclosure, open-loop temperature control is provided in the form of one or more aerogel-based thermal regulation/dissipation layers disposed between the patient's skin and an induction coil of an external inductive transcutaneous power device (referred to herein as an “EPD” for convenience). The one or more thermal regulation/dissipation layer(s) receive and/or absorb heat generated from the induction coil within an aerogel layer, and passively regulate dissipation of heat from the aerogel layer to minimize heat transfer to the patient. In an embodiment, an aerogel layer dissipates heat more slowly than heat is generated by the induction coil such that the aerogel layer slows or otherwise limits a rate of heat dissipation.

Further aspects of the technology described herein will be brought out in the following portions of the specification, wherein the detailed description is for the purpose of fully disclosing embodiments of the technology without placing limitations thereon.

### BRIEF DESCRIPTION OF THE DRAWINGS

The technology described herein will be more fully understood by reference to the following drawings which are for illustrative purposes only:

FIG. 1 shows an exploded perspective view of an embodiment of an EPD with open-loop temperature control according to the technology of the present disclosure.

FIG. 2 shows an assembled perspective view of the EPD of FIG. 1.

FIG. 3 shows a perspective view of an EPD disposed in a band worn around a patient to form a wearable EPD according to the technology of the present disclosure.

FIG. 4A is a partial cross-sectional view of a thickness represented by region A of FIG. 2, showing a schematic view of an embodiment of the EPD of FIG. 2.

FIG. 4B is a partial cross-sectional view of a thickness represented by region A of FIG. 2, showing a schematic view of another embodiment of the EPD.

FIG. 4C is a partial cross-sectional view of a thickness represented by region A of FIG. 2, showing a schematic view of another embodiment of the EPD.

FIG. 4D is a partial cross-sectional view of a thickness represented by region A of FIG. 2, showing a schematic view of another embodiment of the EPD.

FIG. 5A is a partial cross-sectional view of a thickness represented by region B of FIG. 3, showing a schematic view of an embodiment of the wearable EPD.

FIG. 5B is a partial cross-sectional view of a thickness represented by region B of FIG. 3, showing a schematic view of another embodiment of the wearable EPD.

FIG. 5C is a partial cross-sectional view of a thickness represented by region B of FIG. 3, showing a schematic view of another embodiment of the wearable EPD.

### DETAILED DESCRIPTION

In various applications, an IMD may be used to monitor a patient's condition and/or deliver therapy to a patient. By way of example, an IMD may be a device such as an electrical stimulation or treatment device, an implantable pacemaker, a defibrillator, a hearing aid, an organ assistant or replacement device, or other partially or completely implanted medical device.

Embodiments of the present disclosure include systems and methods for providing an EPD with open-loop temperature control for powering/charging, and/or for providing for communication with, an IMD via inductive coupling. Communication may be, for example, unilaterally or bilaterally exchanging signals, data, or commands.

Often, and in particular use cases involving long term or chronic treatment or therapy, the IMD will include a rechargeable power source (e.g., one or more capacitors or batteries) that can extend the operational life of the IMD to weeks, months, or even years beyond that of a non-rechargeable device. When energy stored in the IMD rechargeable power source has been diminished or depleted, the patient may recharge the power source via an EPD. The EPD, which may alternatively be referred to as an “external controller” or “external charging unit”, is configured to be positioned at a location external to the body and adjacent to or near the skin. The EPD wirelessly couples to the IMD by way of inductive coupling between a primary coil in the EPD and a secondary coil in the IMD. Inductive coupling will generally be improved as the primary coil and the secondary coil approach alignment with each other, and also will generally be improved as the primary coil and the secondary coil are brought closer to each other (a distance between is reduced).

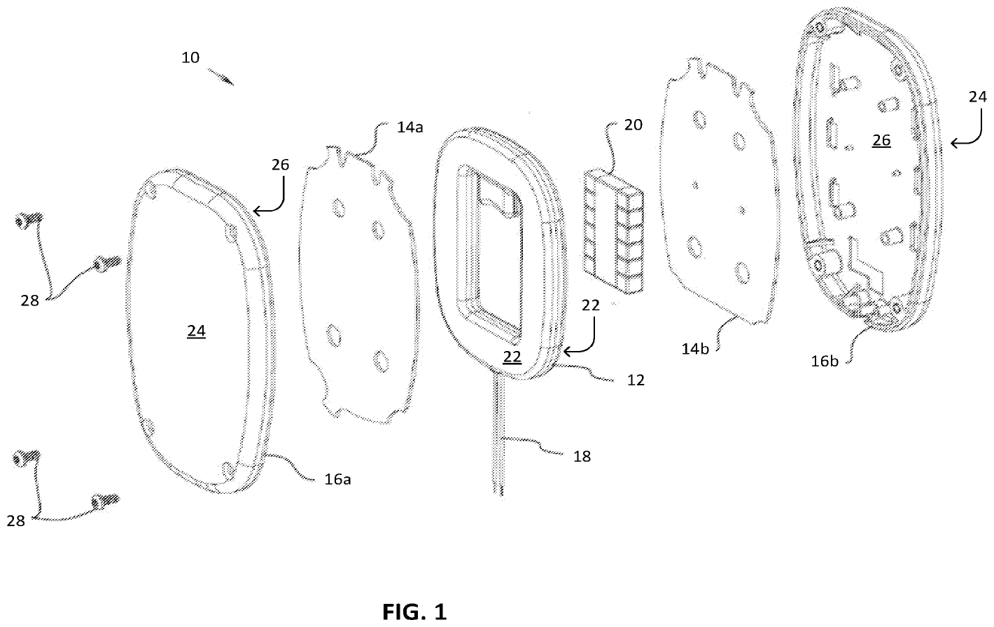

The EPD of the present description, variably detailed in example embodiments shown in FIG. 1 through FIG. 5C, is configured to operate in an open-loop configuration to passively dissipate heat that is inherently generated as a result of the activation of the primary induction coil in the EPD. This controlled heat dissipation is primarily achieved via an aerogel barrier disposed between the induction coil and the patient's skin surface. The aerogel layer is configured to absorb or otherwise receive heat generated from the induction coil and passively regulate (e.g., slow the rate of) dissipation of heat from the EPD to reduce or minimize possible heat transfer to the patient.

While the system and methods of the technology of the present disclosure are configured to operate without closed-loop temperature control, it is appreciated that the systems and methods may also be used to complement or otherwise be incorporated with an EPD having some form of closed-loop control (e.g., temperature feedback).

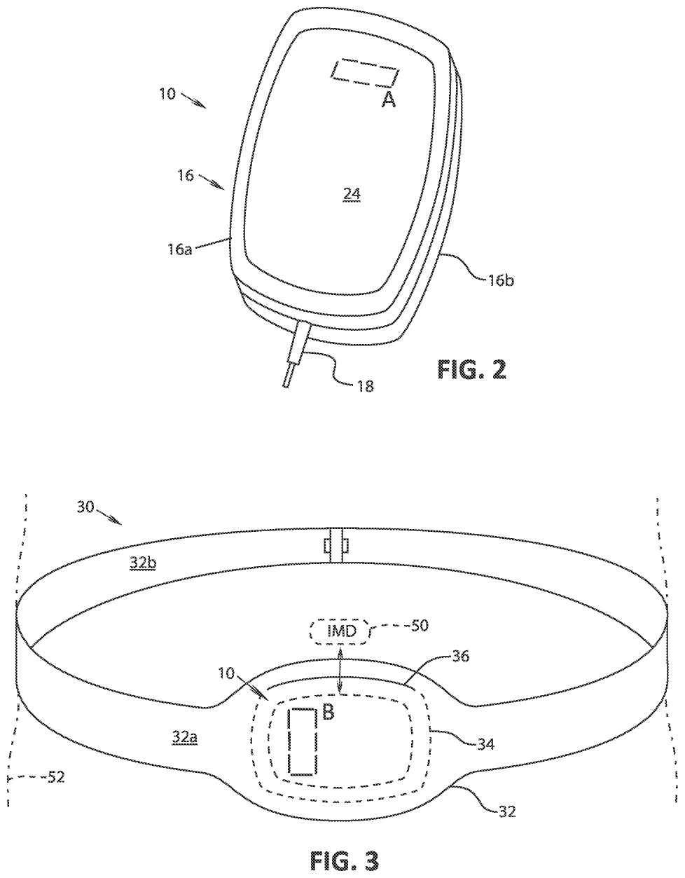

FIG. 1 shows an exploded perspective view of an embodiment of an EPD 10 with open-loop temperature control according to the technology of the present disclosure. FIG. 2 shows a corresponding assembled perspective view of EPD 10 according to an embodiment. The embodiments of an EPD shown in FIG. 1 and FIG. 2 as EPD 10 are provided for illustrative purposes only, and many other shapes and configurations of individual components and an EPD as assembled will be apparent to one of ordinary skill and are within the scope of the present disclosure.

EPD 10 includes a primary induction coil 12 disposed within an upper housing 16a and a lower housing 16b. Induction coil 12 generally includes multiple wire windings (e.g., copper wire) disposed in a loop to surround a common core 20 (e.g., ferrite). The wire windings of induction coil 12, as well as other circuits/logic that may be included with induction coil 12, may be encased in or otherwise contained by an over-mold (e.g., a polymeric or other material molded over). Upper housing 16a and lower housing 16b may be a biocompatible plastic or polymer, although other materials are contemplated. Upper housing 16a and lower housing 16b may be secured together via screw, bolt or other fastener 28 as housing 16 shown in the assembled view in FIG. 2, to encase induction coil 12, core 20, and other associated componentry. Lead wires 18 extend through housing 16, and may be coupled to a connector (e.g., micro USB, USB Mini) for coupling lead wires 18 and induction coil 12 to a power source or controller (not shown). Construction, materials, and components selected for and used with induction coil 12, core 20, lead wires 18, and housing 16 may vary.

In operation, an electric current supplied via lead wires 18 is passed through induction coil 12, creating a magnetic field used in providing power to (or communicating with) an IMD (e.g., IMD 50 in patient 52 as shown in FIG. 3) via inductive coupling. When a current is applied to inductive coil 12 of EPD 10, and EPD 10 is aligned to an extent with a secondary coil (not shown) of IMD 50, electrical current is induced in the secondary coil within the patient. Circuitry (not shown) associated with the IMD can use the current to charge a rechargeable power source (e.g., a battery) within the IMD, and may also use the current to power the IMD and/or to communicate with EPD 10. Inductive coupling inherently generates heat at induction coil 12, emanating outward from surfaces 22 (FIG. 1). Because of the proximity of induction coil 12 to the patient, such heat may irritate or harm a patient, such as when EPD 10 is worn for an extended period of time.

Accordingly, one or more passive heat dissipation layer(s) 14a/14b are disposed between induction coil 12 and a patient to provide open-loop temperature control with respect to EPD 10. Heat dissipation layer(s) 14a/14b may be constructed from a single material or multiple materials. For example, heat dissipation layer 14a or 14b may include an aerogel layer disposed adjacent to or between layers of other material(s), or heat dissipation layer 14a or 14b may include multiple aerogel layers interspersed between layers of other material(s), or heat dissipation layer 14a or 14b may include multiple aerogel layers stacked one against another. When multiple aerogel layers are used, each aerogel layer may have different physical properties and different heat dissipation profiles. Aerogel heat dissipation layer(s) 14a/14b may also include or be integrated with one or more non-aerogel insulative materials or layers.

Heat dissipation layer 14a and/or 14b are configured to receive (e.g., absorb) heat generated from induction coil 12, and can therefore act as a heat barrier between induction coil 12 and the patient's skin surface.

In an embodiment, heat dissipation layer 14a and/or 14b includes a silica aerogel (e.g., Enova® aerogel by Cabot Corporation). However, other aerogels, or combinations of aerogels and other materials (e.g. fiberglass) may be employed to form an aerogel fabric or even rigid structure or material. In the case of an aerogel formed as a rigid structure or material, such structure or material may serve to form a substitute for one or more of upper or lower housings 16a/16b.

For purposes of this description, “aerogel” is defined as a synthetic, porous, ultralight material derived from a gel, in which the liquid component of the gel has been replaced with a gas, and the combination of high porosity and small pores provides the aerogel with low density and low thermal conductivity.

In an embodiment, heat dissipation layer 14a and/or 14b includes an aerogel fabricated by way of a “sol-gel process”, in which nanoparticles suspended in a liquid solution (“sol-”) are invoked to interconnect and form a continuous, porous, nanostructured network of particles across the volume of the liquid medium (“gel”).

An aerogel may be fabricated as a silicon alkoxide gelation, involving the reaction of a silicon alkoxide with water in a solvent such as ethanol or acetone, in the presence of basic, acidic, and/or fluoride-containing catalyst(s). In this technique, a silicon alkoxide (e.g., tetramethoxysilane or tetraethoxysilane) serves as the source for the silica, water acts as a reactant to help join the alkoxide molecules together, and a catalyst (e.g., ammonium hydroxide or ammonium fluoride) facilitates the rate of underlying chemical reactions.

Because of the consistency of the aerogel, the aerogel may be disposed within or between cover materials, or a “blanket,” to retain the aerogel in its intended shape and/or inhibit the aerogel from breaking apart or disintegrating.

The thickness and consistency of heat dissipation layer(s) 14a/14b may vary by application and expected power delivery of induction coil 12. Heat dissipation layer(s) 14a/14b may be very thin to provide proper heat dissipation/regulation without unduly affecting the induction of power from EPD 10 to an IMD. In an embodiment, a thickness of heat dissipation layer(s) 14a/14b is less than 12.7 mm (0.5 in), and preferably less than 6.35 mm (0.25 in), more preferably less than 3.175 mm (0.125 in). In one embodiment, the thickness of the heat dissipation layer(s) 14a/14b ranges from about 2.54 mm (0.1 in) and 3.175 mm (0.125 in).

While the aerogel/heat dissipation layer is shown as a pair of thin sheets 14a/14b in FIG. 1, it is appreciated that heat dissipation layer(s) 14a/14b may be manufactured in a number of different forms for creating a heat barrier in at least one direction. For example, EPD 10 in FIG. 1 details a pair of thin sheets having a shape and cutouts formulated according to the shape and configuration of upper and lower housings 16a/16b. In an embodiment, heat dissipation layer(s) 14a/14b are adhered to or otherwise secured to respective inner surfaces 26 of upper and lower housings 16a/16b or to opposing outer surfaces 22 of induction coil 12. In an embodiment, heat dissipation layer(s) 14a/14b are adhered to or otherwise secured to outer surfaces 24 of upper and lower housings 16a/16b.

Alternatively or additionally to heat dissipation layer(s) 14a/14b formed as components and assembled into EPD 10, heat dissipation layer 14a and/or 14b can be a coating disposed on any one or more of: one or both surfaces 22 of induction coil 12; outer surface 24 of upper housing 16a; outer surface 24 of lower housing 16b; inner surface 26 of upper housing 16a; inner surface 26 of lower housing 16b; or over all surfaces of housing 16 as assembled. Such coating can, for example, fully or partially encase induction coil 12, upper housing 16a, lower housing 16b, and/or housing 16 as a whole. Such coating can provide thermal regulation or a thermal barrier in at least one direction (at least towards patient 52). Such coating may be fabricated via any number of processes (e.g., deposition, spray coating, dip, or over-mold).

The heat dissipation/thermal regulation features of the systems and methods of the present description may also be incorporated or integrated into a wearable device. FIG. 3 shows a perspective view of an embodiment of a wearable EPD 30. In embodiments, the wearable EPD 30 includes an EPD 10 that is disposed in a band 32 that can be worn around a portion of patient 52. In at least some embodiments, the wearable EPD 30 can be worn to couple to an IMD 50. For illustration, IMD 50 is shown in FIG. 3 as a simple shape disposed in a given position within patient 52.

Band 32 has outer surface 32a and inner surface 32b. Inner surface 32b is adjacent patient 52, such as contacting skin of patient 52 or contacting clothing worn by patient 52. In an embodiment, band 32 is structured to enable the wearable EPD 30 to be worn such that the primary induction coil 12 of EPD 10 (see FIG. 1) couples to a coil of an implanted IMD 50 (e.g., implanted in or near the heart, at the spine, in the pelvic area, in the buttocks, in an extremity, or at a subcutaneous location) of patient 52. Wearable EPD 30 may be any number of configurations (e.g., a chest belt, head band, waist belt, arm band, or patch) designed for coupling at or near a skin location in proximity to the location of the IMD.

Band 32 may be formed from soft material (e.g., including fabric), bendable material (e.g., including silicone), or rigid material (e.g., including hard plastic). In an embodiment, EPD 10 is disposed within a pocket 34 of band 32. The pocket 34 can be structured to receive and retain the EPD 10 with the band 32, such that the EPD 10 may be inserted into, retained in and/or removed from pocket 34 via an opening 36. In variations, other configurations or structures can provided with band 32 and/or EPD 10 to enable the EPD 10 to be retained or otherwise provided with the band 32. For example, the band 32 can include a flap, drawer, or other accessible space where the EPD 10 can be received, retained and removed.

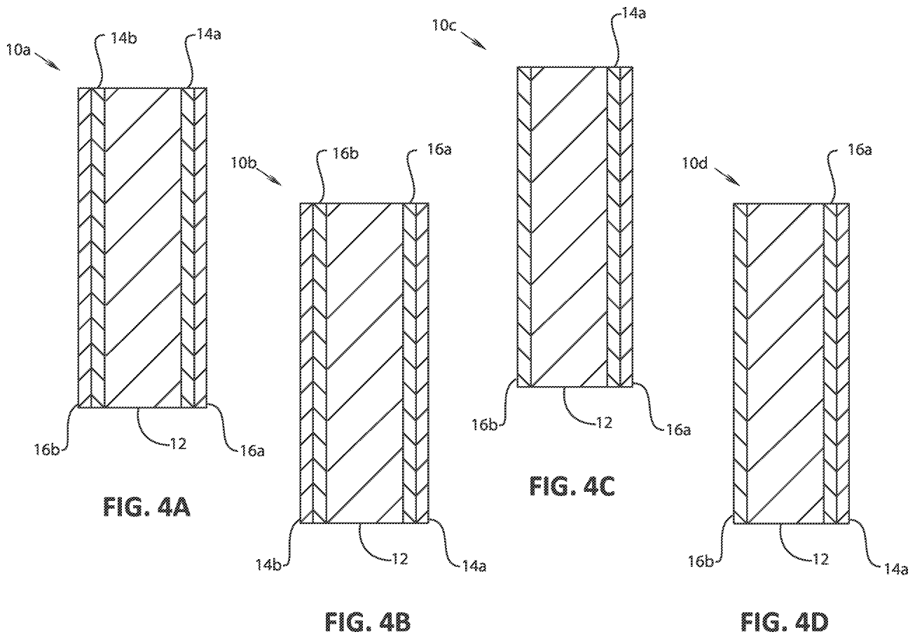

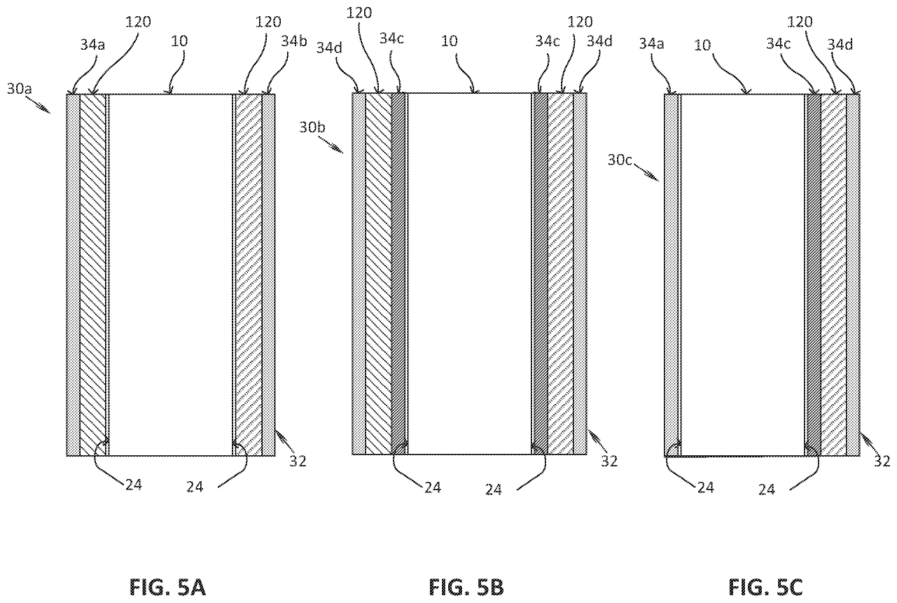

As described in further detail below, embodiments may include various configurations for including one or more heat dissipation layer(s) 14a/14b with EPD 10 or wearable EPD 30. FIG. 4A through FIG. 4D illustrate alternative configurations for providing heat dissipation layer(s) 14a/14b within EPD 10. As an addition or variation to embodiments of FIG. 4A through FIG. 4D, FIG. 5A through FIG. 5C illustrate alternative configurations providing heat dissipation layer(s) 120 within the wearable EPD 30, as an addition or alternative to heat dissipation layers 14a/14b of EPD 10. In any of the disclosed configurations, heat dissipation layer(s) 14a/14b, 120 may be in the form of a sheet, coating, or other structure disclosed herein that is coupled to or integrated into or disposed adjacent to one or more surfaces of the various components.

Referring now to FIGS. 4A-4C, partial cross-sectional views of a thickness represented by region A of FIG. 2 are shown, illustrating schematic views of various embodiments of EPD 10 (EPD 10a/10b/10c/10d in FIGS. 4A/4B/4C/4D, respectively).

FIG. 4A illustrates EPD 10a including two heat dissipation layers 14a, 14b disposed on opposing sides of induction coil 12, between induction coil 12 and inner surfaces 26 of respective upper and lower housings 16a/16b.

FIG. 4B illustrates EPD 10b including two heat dissipation layers 14a, 14b disposed on opposing outer surfaces 24 of respective upper and lower housings 16a/16b.

FIG. 4C illustrates EPD 10c including a single heat dissipation layer 14a disposed between induction coil 12 and inner surface 26 of upper housing 16a, and with no heat dissipation layer disposed adjacent to lower housing 16b. For use in this configuration, upper housing 16a is disposed facing patient 52 to provide thermal regulation for heat generated from induction coil 12 in the direction of patient 52.

FIG. 4D illustrates EPD 10d including a single heat dissipation layer 14a is disposed on outer surface 24 of upper housing 16a, and with no heat dissipation layer disposed adjacent to lower housing 16b. For use in this configuration, upper housing 16a is disposed facing patient 52 to provide thermal regulation for heat generated from induction coil 12 in the direction of patient 52.

Referring now to FIGS. 5A-5C, partial cross-sectional views of a thickness represented by region B of FIG. 3 are shown, representing schematic views of various embodiments of wearable EPD 30 (wearable EPDs 30a/30b/30c in FIGS. 5A/5B/5C, respectively). As shown, the wearable EPD 30 includes EPD 10 in a pocket 34 of band 32. Pocket 34 fully or partially supports EPD 10 at or along perimeter edges of the EPD 10, so as to retain the EPD 10 with the band 32 in a selected or operative position. Pocket 34 includes at least two sides 34a, 34b. In an example, pocket 34 may be formed by a single length of a material that is folded to form both sides 34a, 34b. In another example, the pocket 34 may be formed by one or more lengths of material that together form the respective side 34a or 34b. Materials used to form pocket 34 may include, for example, mesh, fabric, polymer, fiber weave, polymer, plastic, or other types of flexible material(s) that collectively form a pocket.

In FIGS. 5A-5C, one or more heat dissipation layer(s) are provided within wearable EPD 30. In some embodiments, the wearable EPD 30 includes one or more heat dissipation layers 120 that are provided external to the EPD 10. Depending on implementation, the EPD 10 of wearable EPD 30 may or may not itself also include heat dissipation layers 14a/14b or materials. For example, in some embodiments, the EPD 10 of wearable EPD 30 includes heat dissipation layers 14a/14b in configurations such as described with FIG. 4A-4C, while in variations, the EPD 10 of wearable EPD 30 includes no heat dissipation layers 14a/14b or material. In FIG. 5A, wearable EPD 30a includes two heat dissipation layers 120 that are disposed within pocket 34 of band 32, and each heat dissipation layer 120 is adjacent to one of opposing outer surfaces 24 of EPD 10. In some embodiments of this configuration, one or more heat dissipation layers 120 may be sewn, adhered, or otherwise attached onto respective sides 34a/34b of pocket 34. For example, depending on implementation, wearable EPD 30 may include one or two (or more) heat dissipation layers 120, separate from the EPD 10.

In FIG. 5B, wearable EPD 30b includes two heat dissipation layers 120 that are disposed within pocket 34 of band 32. Each heat dissipation layer 120 is near one of opposing outer surfaces 24 of EPD 10 but there is a separation layer 34c of pocket 34 between EPD 10 and each dissipation layer 120. Accordingly, heat dissipation layers 120 have on one side separation layer 34c and on the other side an outer layer 34d of pocket 34. In an embodiment of this configuration, heat dissipation layers 120 may be in a raw gel form that is contained between separation layer 34c and outer layer 34d.

In FIG. 5C, wearable EPD 30c includes a single heat dissipation layer 120 disposed on one side of EPD 10 between separation layer 34c and outer layer 34d. On the other side of EPD 10, no separation layer is included. In an embodiment, a heat dissipation layer 120 is disposed on that other side of EPD 10 within pocket 34 directly adjacent to EPD 10 without a separation layer. In the configuration illustrated in FIG. 5C, heat dissipation layer 120 may be in a raw gel form that is contained between layers 32c/32d. For use in the configuration illustrated in FIG. 5C, the side of pocket 34 including heat dissipation layer 120 is disposed facing patient 52 to provide thermal regulation for heat generated from induction coil 12 in the direction of patient 52.

It is also appreciated that heat dissipation layers may be disposed on one or more of the outer surfaces of 32a and 32b of band 32 (not shown).

From the description herein, it will be appreciated that the present disclosure encompasses multiple embodiments which include, but are not limited to, the following:

1. An apparatus for remotely powering an implantable medical device (IMD) positioned at a target treatment location within a patient, the apparatus configured to be positioned at or near an external skin surface of the patient in proximity to the target treatment location. The apparatus comprises: an induction coil which, when placed in proximity to the IMD, forms an inductive transcutaneous power link with the IMD such that when supplied with a current, the induction coil inductively and transcutaneously delivers power to the IMD; and an aerogel layer disposed between the induction coil and the patient's skin surface; wherein the aerogel layer is configured to receive heat generated from the induction coil and passively regulate dissipation of the heat from the aerogel layer to minimize heat transfer to the patient. The induction coil may be disposed within a housing.

2. A method for providing treatment to a target location within the body of a patient, comprising: delivering an implantable medical device (IMD) to a target location; positioning an external charging device at an external location at or near a skin surface of the patient in proximity to the target location, the external charging device comprising an induction coil; inductively delivering power to the IMD via an inductive transcutaneous power link established between the external charging device and the IMD; receiving heat generated from the induction coil within an aerogel layer disposed between the induction coil and the patient's skin surface; and passively regulating dissipation of the heat from the aerogel layer to minimize heat transfer to the patient. The induction coil may be disposed within a housing.

3. A wearable device for remotely powering an implantable medical device (IMD) located at a target treatment location within a patient, comprising: a pocket configured to be located at an external location at or near a skin surface of the patient in proximity to the target treatment location; a band configured to secure to the patient at or near said external location, the band defining or including the pocket; an induction coil disposed within the pocket; wherein the induction coil, when placed in proximity to the IMD, forms an inductive transcutaneous power link with the IMD such that when supplied with a current, the induction coil inductively and transcutaneously delivers power to the IMD; and an aerogel layer disposed between the induction coil and the patient's skin surface; wherein the aerogel layer is configured to receive heat generated from the induction coil and passively regulate dissipation of the heat from the aerogel layer to minimize heat transfer to the patient. The induction coil may be disposed within a housing.

4. The apparatus, method, or device of any preceding or subsequent embodiment, wherein the aerogel layer slows dissipation of heat out of the aerogel layer to a rate that is less than a rate of heat generated by operation of the induction coil.

5. The apparatus, method, or device of any preceding or subsequent embodiment, wherein the aerogel layer is part of an open control loop for regulating heat dissipated from the induction coil.

6. The apparatus, method, or device of any preceding or subsequent embodiment, wherein the aerogel layer comprises a silica aerogel.

7. The apparatus, method, or device of any preceding or subsequent embodiment, wherein the aerogel layer is disposed between an outer surface of the induction coil and an inner surface of the housing.

8. The apparatus, method, or device of any preceding or subsequent embodiment, wherein the aerogel layer is disposed on an outer surface of the housing.

9. The apparatus, method, or device of any preceding or subsequent embodiment, wherein the aerogel layer comprises a coating applied to one or more surfaces of the housing and/or the induction coil.

10. The apparatus, method, or device of any preceding or subsequent embodiment, wherein the aerogel layer comprises a coating that encases the outer surface of the housing and/or an outer surface of the induction coil.

11. The apparatus, method, or device of any preceding or subsequent embodiment, wherein the aerogel layer is disposed directly adjacent to an outer surface of the induction coil.

12. The apparatus, method, or device of any preceding or subsequent embodiment, wherein the aerogel layer is disposed directly adjacent to an outer surface of the housing.

13. The method or apparatus of any preceding or subsequent embodiment, wherein the aerogel layer comprises a film or thin film.

14. The method or apparatus of any preceding or subsequent embodiment, wherein the aerogel layer is disposed between two fabric layers.

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural referents unless the context clearly dictates otherwise. Reference to an object in the singular is not intended to mean “one and only one” unless explicitly so stated, but rather “one or more.”

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The foregoing description of various embodiments of the technology of the present disclosure has been presented for purposes of illustration and description. It is not intended to limit the technology of the present disclosure to the precise forms disclosed. Many modifications, variations and refinements will be apparent to practitioners skilled in the art. Further, those skilled in the art will recognize, or be able to ascertain using no more than routine experimentation, numerous equivalents to the specific devices and methods described herein. Such equivalents are considered to be within the scope of the technology of the present disclosure and are covered by the appended claims below.

Elements, characteristics, or acts from one embodiment can be readily recombined or substituted with one or more elements, characteristics or acts from other embodiments to form numerous additional embodiments within the scope of the technology of the present disclosure. Moreover, elements that are shown or described as being combined with other elements, can, in various embodiments, exist as standalone elements. Hence, the scope of the technology of the present disclosure is not limited to the specifics of the described embodiments but is instead limited solely by the appended claims.

What is claimed is:

1. An apparatus for remotely powering an implantable medical device (IMD) positioned at a target treatment location within a patient, the apparatus configured to be positioned at or near an external skin surface of the patient in proximity to the target treatment location comprising:
an induction coil which, when placed in proximity to the IMD, forms an inductive transcutaneous power link with the IMD such that when the induction coil is supplied with a current, the induction coil inductively and transcutaneously delivers power to the IMD; and
an aerogel layer disposed between the induction coil and the patient's skin surface;
wherein the aerogel layer is configured to receive heat generated from the induction coil and regulate heat dissipation from the aerogel layer to minimize heat transfer to the patient.

2. The apparatus of claim 1, wherein the aerogel layer slows dissipation of heat out of the aerogel layer to a rate that is less than a rate of heat generated by operation of the induction coil.

3. The apparatus of claim 2, wherein the aerogel layer is part of an open control loop for regulating heat dissipated from the induction coil.

4. The apparatus of claim 1, wherein the aerogel layer comprises a silica aerogel.

5. The apparatus of claim 1, further comprising a housing surrounding the induction coil.

6. The apparatus of claim 5, wherein the aerogel layer is disposed between an outer surface of the induction coil and an inner surface of the housing.

7. The apparatus of claim 5, wherein the aerogel layer is disposed on an outer surface of the housing.

8. The apparatus of claim 5, wherein the aerogel layer comprises a coating applied to one or more surfaces of the housing and/or the induction coil.

9. The apparatus of claim 8, wherein the coating encases an outer surface of the housing and/or an outer surface of the induction coil.

10. A method for providing treatment to a target location within a body of a patient, the method comprising:
delivering an implantable medical device (IMD) to the target location;
positioning an external charging device at or near an external skin of the patient in proximity to the target location, the external charging device comprising an induction coil;
inductively delivering power to the IMD via an inductive transcutaneous power link established between the external charging device and the IMD;
receiving heat generated from the induction coil within an aerogel layer disposed between the induction coil and the external surface of skin; and
passively regulating dissipation of the heat from the aerogel layer to minimize heat transfer to the patient.

11. The method of claim 10, wherein the aerogel layer slows dissipation of heat out of the aerogel layer to a rate that is less than a rate of heat generated by operation of the induction coil.

12. A wearable device for remotely powering an implantable medical device (IMD) located at a target treatment location within a patient, comprising:
a pocket configured to be located at an external location at or near a skin surface of the patient in proximity to the target treatment location;
a band configured to secure to the patient at or near said external location, the band defining or including the pocket;
an induction coil disposed within the pocket, wherein the induction coil, when placed in proximity to the IMD, forms an inductive transcutaneous power link with the IMD such that when supplied with a current, the induction coil inductively and transcutaneously delivers power to the IMD;
a housing surrounding the induction coil; and
an aerogel layer disposed between the induction coil and the patient's skin surface; wherein the aerogel layer is configured to receive heat generated from the induction coil and passively regulate dissipation of the heat from the aerogel layer to minimize heat transfer to the patient.

13. The wearable device of claim 12, wherein the aerogel layer slows dissipation of heat out of the aerogel layer to a rate that is less than a rate of heat generated by operation of the induction coil.

14. The wearable device of claim 12, wherein the aerogel layer is disposed on an outer surface of the housing.

15. The wearable device of claim 12, wherein the aerogel layer is disposed between an outer surface of the induction coil and an inner surface of the housing.

16. The wearable device of claim 12, wherein the aerogel layer comprises a coating applied to one or more surfaces of the housing and/or the induction coil.

17. The wearable device of claim 16, wherein the coating encases an outer surface of the housing and/or the induction coil.

18. The wearable device of claim 12, wherein the pocket is configured to secure the housing in such a way that the housing may be removed and replaced.

19. The wearable device of claim 18, wherein the pocket comprises a flexible material.

20. The wearable device of claim 18, wherein the pocket comprises a rigid material.
